(19) United States
(12) Patent Application Publication
(10) Pub. No.: US 2014/0155435 A1
(43) Pub. Date: Jun. 5, 2014

(54) Kit for Treatment of Upper Gastrointestinal Tract Conditions

(71) Applicant: THE PROCTER & GAMBLE COMPANY, Cincinnati, OH (US)

(72) Inventors: Will Howard Mitchell, Loveland, OH (US); Linda Carol Jones, Aurora, IN (US); John Stephen Eadicicco, West Chester, OH (US); Beth Waggoner Russell, Goshen, OH (US)

(73) Assignee: The Procter & Gamble Company, Cincinnati, OH (US)

(21) Appl. No.: 13/705,215

(22) Filed: Dec. 5, 2012

(51) Int. Cl. A61K 31/4439

(52) U.S. Cl. CPC A61K 31/4439
USPC 514/338

(57) ABSTRACT

Disclosed herein are kits useful for facilitating compliance with a treatment regimen. In one embodiment, the kits are useful for compliance with a defined method of treatment which utilizes a composition comprising a gastric acid secretion inhibitor. In an alternative or additional embodiment, the kits comprise an outer container and at least two inner containers, wherein each of the inner containers comprise, independently, a plurality of unit doses of a composition comprising a therapeutically effective amount of a gastric acid secretion inhibitor.

### CROSS REFERENCE TO RELATED APPLICATION

This application is a continuation of U.S. application Ser. No. 10/922,260, filed on Aug. 19, 2004; which claimed the benefit of U.S. Provisional Application No. 60/496,423, filed Aug. 20, 2003.

### FIELD OF THE INVENTION

The invention further relates to a kit for facilitating user compliance with the described regimens of treatment for upper gastrointestinal conditions. Specifically, the kits relate to facilitation of user compliance in treatments utilizing a gastric acid secretion inhibitor.

### BACKGROUND OF THE INVENTION

User compliance with treatment regimens remains a significant healthcare issue. Compliance is further significant when medications are approved for over-the-counter (OTC) sale, as this provides the user with increased access to medications. Even further, compliance with upper gastrointestinal conditions, for example frequent heartburn, presents some particularly unique issues. Because of the pain and unpleasantness of the symptoms, users may require little encouragement to begin a treatment regimen. In some users, however, symptoms are persistent or, in contrast, transient. If may be desirable to inform these users regarding the optimal course of treatment for the condition.

Frequent heartburn results from gastric acid being released into the lower esophagus. The user usually experiences a burning chest pain, which begins behind the breastbone and moves upward to the neck and throat. Gastrointestinal Disease, Pathophysiology, Diagnosis and Management, 5^{th} Ed., Vol. 1, B. Scharschmidt and M. Feldman, Eds., pp. 378-79 (1993). Proton pump inhibitors (PPIs) are a class of pharmaceutical compounds that effectively prevent or inhibit gastric acid secretion by inhibiting the H⁺/K⁺-ATP enzyme system. Drug Facts and Comparisons, Cada et al. Eds., pp. 1137-38 (May 2003). Omeprazole is one example of a PPI that has proven effective in the prevention and treatment of upper gastrointestinal conditions, including frequent heartburn, GERD, gastritis, gastric ulcer and duodenal ulcer. See e.g., U.S. Pat. Nos. 4,508,905; 4,738,974; and 5,900,424.

A composition containing omeprazole has recently been approved for OTC sale for the treatment of frequent heartburn. This raises the aforementioned complex user compliance issue.

A number of references describe treatment regimens for certain medications and seek to increase user compliance therewith. For example, many are directed to the administration of bisphosphonates, which belong to the class of compounds known as bone resorption inhibitors. See e.g., U.S. Pat. Nos. 4,761,406; 5,616,560; 5,994,329. Bisphosphonates are prescribed for the treatment of chronic conditions, such as osteoporosis, in which there is a continual loss of bone due to resorption. Strict adherence to these dosing regimens is necessary for effective treatment, and user compliance is an important concern. See e.g., U.S. Pat. No. 5,366,965 and Drug Facts and Comparisons, Cada et al., Eds., p. 584 (May 2003, updated monthly).

The above dosing regimens can be either continuous or cyclic. Continuous dosing regimens involve administration of relatively low doses of bisphosphonates at regular intervals. One problem with continuous dosing of bisphosphonates, however, is that the body adjusts to the attempts to regulate bone resorption, and may counteract any gain in bone mass. Cyclic dosing regimens were developed to address this problem, and require administration of higher doses of bisphosphonates during a given time interval, followed by a rest period during which no drug is administered. Without intending to be limited by theory, the physiological basis for the rest period is to uncouple bone formation and resorption by selectively inhibiting the resorption phase of bone remodeling without appreciably affecting the formation phase. Disorders of Bone and Mineral Metabolism, Fredric L. Coe and Murry J. Favus, Eds., 866-67 (1992); U.S. Pat. No. 4,761,406. As such, the success of these therapies are uniquely dependent upon the prescribed rest period.

Several references also teach kits that are designed to increase user compliance with these and other treatment regimens. See e.g., U.S. Pat. Nos. 4,534,468; 4,889,237; 5,833,072; and U.S. Patent Publication 2001/0044427 A1.

However, there is a continuing need to address the more complex user compliance issues that arise from over-the-counter sale of medication for the treatment of gastrointestinal conditions. For example, the aforementioned dosing regimens and kits fail to address the problem of encouraging responsible use of over-the-counter medications to treat acute symptoms of gastrointestinal disorders.

The present invention provides a convenient kit optionally designed to facilitate user compliance with a flexible dosing regimen. The treatment regimen provides for dosing periods during which a sufficient number of doses of a gastric acid secretion inhibitor are administered to provide relief from symptoms. The present invention provides for dosing according to a discontinuous schedule. In a discontinuous schedule, each dosing period is followed by an evaluation period, during which the user can self-evaluate the occurrence and severity of symptoms. If the user determines that it is necessary to begin a new dosing period, the user may do so at any time following this evaluation period. If, however, the user feels the need to begin a new dosing period before the evaluation period has passed, or to take more than the recommended number of doses, then the user may, for example, choose to seek professional medical advice.

The kits are designed to encourage compliance with the dosing and to the evaluation periods. In one embodiment, multiple, individually-packaged dosing regimens are contained in a single kit. Each individual package contains a sufficient number of doses of a gastric acid secretion inhibitor for one dosing period, together with a set of instructions, including optionally motivational text. To begin a new dosing period, the user therefore opens a separate package containing instructions, including optionally motivational text. The inclusion of instructions in individually packaged dosing regimens, with multiple dosing regimens contained in a single kit, is a unique feature of the present invention, and serves as a signal to remind the user that a new treatment regimen may not be undertaken until sufficient time for self-evaluation of symptoms has passed.

### SUMMARY OF THE INVENTION

The present invention further relates to kits useful for facilitating compliance with a treatment regimen.

In one embodiment, the kits comprise:
(a) an outer container;
(b) at least two inner containers, wherein each inner container comprises a plurality of unit doses of a composition comprising a therapeutically effective amount of a gastric acid secretion inhibitor; and
(c) instructions to facilitate compliance with a method for treating an upper gastrointestinal tract condition in a mammalian subject in need thereof, wherein the method comprises administering the to the mammalian subject in accordance with a discontinuous schedule, wherein:
   (i) the discontinuous schedule comprises a first dosing period, a first evaluation period, and a second dosing period;
   (ii) the first evaluation period is at least about 2 days and is subsequent to the first dosing period and precedes the second dosing period; and
   (iii) during each of the first and second dosing periods, independently, a plurality of unit doses of the composition is administered to the mammalian subject.

In another embodiment, the kits comprise:
(a) an outer container;
(b) at least two inner containers, wherein each inner container comprises a plurality of unit doses of a composition comprising a therapeutically effective amount of omeprazole; and
(c) instructions to facilitate compliance with a method for treating an upper gastrointestinal tract condition in a mammalian subject in need thereof, wherein the method comprises administering the composition to the mammalian subject in accordance with a discontinuous schedule, wherein:
   (i) the discontinuous schedule comprises a first dosing period,
   (ii) the first evaluation period is at least about 2 days and is subsequent to the first dosing period and precedes the second dosing period;
   (iii) during each of the first and second dosing periods, independently, a plurality of unit doses of the composition is administered to the mammalian subject; and
   (iv) when the omeprazole is an omeprazole salt, the omeprazole salt is selected from the group consisting of omeprazole lithium salts, omeprazole sodium salts, omeprazole potassium salts, omeprazole magnesium salts, omeprazole calcium salts, and mixtures thereof.

In yet another embodiment, the kits comprise an outer container and at least two inner containers, wherein each of the inner containers comprise, independently, a plurality of unit doses of a composition comprising a therapeutically effective amount of a gastric acid secretion inhibitor.

These and other embodiments of the invention are described herein.

### DETAILED DESCRIPTION OF THE FIGURES

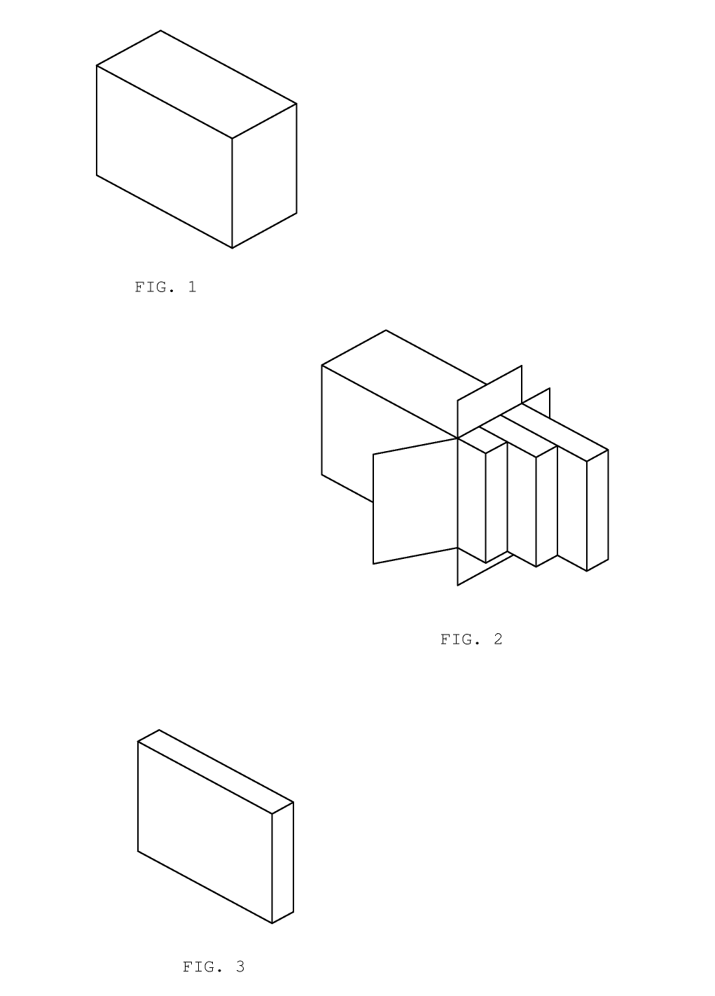

Presented for convenience, FIG. 1 is an illustrative example of an outer container in accordance with the present invention.

Also presented for convenience, FIG. 2 is an illustrative example of the outer container containing three separate inner containers. In an embodiment herein, the inner containers each comprise substantially similar instructions.

Also presented for convenience, FIG. 3 is an illustrative example of one of the separate inner containers also depicted in FIG. 3.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments or individual features are disclosed. As will be apparent to the ordinarily skilled practitioner, all combinations of such embodiments and features are possible and can result in preferred executions of the present invention.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

The methods and kits herein utilize a composition comprising a gastric acid secretion inhibitor. As used herein, gastric acid secretion inhibitor refers to any compound possessing a cytoprotective and/or gastric acid anti-secretory effect, including, but not limited to, proton pump inhibitors such as omeprazole, esomeprazole, lansoprazole, pantoprazole, and rabeprazole, as well as histamine H₂-antagonists such as ranitidine, cimetidine, nazatidine and famotidine, and mixtures of any of the above. In one embodiment, the methods and kits utilize omeprazole. To illustrate, the omeprazole may be either omeprazole or an omeprazole salt. Examples of salts include omeprazole lithium salts, omeprazole sodium salts, omeprazole potassium salts, omeprazole magnesium salts, omeprazole calcium salts, or mixtures thereof. In another embodiment, the omeprazole salt is selected from omeprazole magnesium salts and omeprazole calcium salts, with omeprazole magnesium salts being among the most preferred. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

Optionally, the composition, or kit, may contain active components other than the gastric acid secretion inhibitor. For example, an antacid may be useful herein. As an example, a composition utilized herein may comprise a gastric acid secretion inhibitor as well as an antacid. As another example, the kits may comprise distinct compositions, wherein a first composition comprises a gastric acid secretion inhibitor and a second composition comprises an antacid. See e.g., U.S. Pat. Nos. 5,385,739; 5,840,737; 6,090,412; 6,183,776; 6,489,346; and 6,551,621; WO 97/25066; WO 00/26185; and EP 0,338,861.

The composition described herein comprises the gastric acid secretion inhibitor, optionally but preferably with a therapeutically acceptable carrier. “Therapeutically acceptable carrier,” as used herein, refers to one or more compatible solid or liquid filler diluents or encapsulating substances which are suitable for administration to a mammal in need thereof, preferably a human. The term “compatible,” as used herein, means that the solid or liquid filler diluents are capable of being combined with the gastric acid secretion inhibitor and with each other, in a manner such that there is no interaction that would substantially reduce the pharmaceutical efficacy of the composition under ordinary use situations. Such therapeutically effective carriers are selected according to criteria well-known to those ordinarily skilled in the art. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

The composition of the present invention is preferably administered in the form of unit doses. As used herein, a “unit dose” contains a therapeutically effective amount of the gastric acid secretion inhibitor, and is suitable for administration to a mammal in need thereof, preferably a human, in a single dose. As an example, each unit dose of the composition may be in the form of either a tablet or capsule, optionally wherein the form comprises some enteric coating (for example, an enteric coating surrounding the tablet or capsule itself or utilizing microencapsulation of the gastric acid secretion inhibitor). As an example, a unit dose of a composition comprising omeprazole may be in the form of a capsule. As another example, a unit dose of a composition comprising an omeprazole magnesium salt may be in the form of a tablet. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

As used herein, the term “therapeutically effective amount,” with reference to a gastric acid secretion inhibitor, means that amount of the inhibitor sufficient to provide a significant improvement of the relevant gastrointestinal condition in a mammal, preferably a human, in need of treatment, yet low enough to avoid adverse effects (such as toxicity, irritation, or allergic response), commensurate with a reasonable benefit/risk ratio when used in the manner of the present invention. The specific “therapeutically effective amount” will vary with such factors as the particular condition being treated, the physical condition of the user, the duration of treatment, the nature of concurrent therapy (if any), the specific dosage form to be used, the carrier employed, the solubility of the dose form, and the particular dosing regimen. The most preferred therapeutically effective amount is from about 5 mg to about 35 mg, alternatively from about 10 mg to about 25 mg of the gastric acid secretion inhibitor in each unit dose. See e.g., U.S. Pat. Nos. 4,255,431; 4,508,905; 4,738,974; 4,636,499; 5,900,424; 4,786,505; 4,853,230; 5,690,960; 5,817,338; and 5,753,265.

The mammals treated herein include, but are not limited to, humans. The most preferred embodiment of which is humans in need of treatment for upper gastrointestinal tract conditions. As used herein, upper gastrointestinal tract conditions include, but are not limited to, gastroesophageal reflux disease (GERD), erosive esophagitis, gastritis, gastric ulcers, duodenal ulcers, heartburn (including frequent heartburn), indigestion, posterior laryngitis, hypersecretory conditions, such as Zollinger-Ellison syndrome, multiple endocrine adenomas and systemic mastocytosis, and other diseases or disorders in which cytoprotective and/or gastric acid anti-secretory effect is desirable, such as in users with gastrinomas or acute upper gastrointestinal bleeding, or to enhance the efficacy of pancreatin.

### Kits of the Present Invention

The present invention relates to kits of various embodiments. In one embodiment, the kit comprises a plurality of unit doses of the gastric acid secretion inhibitor and instructions for complying with a treatment method as described herein. Alternatively or additionally, the kit comprises an outer container and at least two individual inner containers, each inner container containing at least two unit doses of a therapeutically effective amount of the gastric acid secretion inhibitor.

In a one embodiment herein, the kit comprises the outer container and instructions for complying with a treatment method as described herein. In another preferred embodiment, the outer container is a box. Alternatively or additionally, in another preferred embodiment, each inner container, independently, is a box, sealed pouch or other similar container of a suitable size that fits within the outer container. In a highly preferred embodiment, each inner container is a box. In another highly preferred embodiment each inner container is a box which is substantially similar (for example, with respect to shape, dimensions, or the like) to each of the other inner container(s) contained within the outer container. Alternatively or additionally, each inner container comprises instructions for complying with the treatment method described herein. Thus, in one embodiment, the user of the kit is reminded of such instructions for compliance each time a different inner container is used.

In one embodiment, the outer container contains at least three inner containers. Alternatively or additionally, the outer container contains a number of inner containers which corresponds to the anticipated number of dosing periods which the user will experience (for example, depending upon whether the particular user experiences frequent, or infrequent, upper gastrointestinal tract conditions).

In another embodiment, the kit is comprised of an outer box that contains two inner boxes, each inner box in turn comprising a sufficient number of unit doses of the gastric acid secretion inhibitor to comply with a given dosing period and instructions for complying with the treatment method. In another embodiment, the kit is comprised of an outer box that contains three inner boxes, each inner box in turn comprising a sufficient number of unit doses to comply with one dosing period and instructions for complying with the treatment method.

In another embodiment, the unit doses are contained in a blister pack. Alternatively or additionally, each inner container contains at least one blister pack, wherein each blister pack contains at least one unit dose. In one embodiment, fourteen unit doses are contained in two blister packs, each blister pack containing seven unit doses. In one embodiment of this type, two or three inner containers each comprise two of the blister packs, wherein each of the blister packs comprises seven unit doses. In another embodiment, fourteen unit doses are contained in one blister pack, each blister pack containing fourteen unit doses. In one embodiment of this type, two or three inner containers each comprise one of the blister packs, wherein each of the blister packs comprises fourteen unit doses.

As used herein, the term “instructions” refers to printed material that sets forth a description of how the user is to comply with the methods of the present invention (e.g., the discontinuous schedule). Such instructions may include descriptions through words, pictures, symbols, and/or other visible descriptors. Such direction need not utilize the actual words used herein, for example, “treatment”, “gastrointestinal,” “gastrointestinal tract condition”, “dosing period,” “evaluation period,” or the like, but rather use of words, pictures, symbols, and the like reasonably conveying same or similar meanings are contemplated within the scope of this invention.

Variants in terms of composition used, length of various dosing periods, and the like are set forth above with respect to the method described herein below and are all incorporated into the descriptions of the kits.

### Methods Optionally Used in Accordance with the Present Invention

In certain aspects, the present invention may relate to a method of treating upper gastrointestinal conditions in mammals, preferably in humans, in need of such treatment. The method comprises administering a composition comprising the gastric acid secretion inhibitor in accordance with a discontinuous schedule, which includes a first dosing period, a first evaluation period, and a second dosing period. During each dosing period, a plurality of unit doses of a composition containing a therapeutically effective amount of a gastric acid secretion inhibitor is administered.

“Administering,” “administer,” or the like, as used herein, refers to any means of introducing a therapeutic amount of a gastric acid secretion inhibitor to the subject in need thereof. The most preferred means is oral administration. As used herein, the term “administration,” “administering,” or the like with respect to the mammal means that the mammal is administered, is directed to administer or, with reference specifically to “oral administration,” or “orally administering,” ingests or is directed to ingest, one or more compositions described herein. Wherein the mammal is directed to ingest one or more of the compositions, such direction may be that which instructs and/or informs the user that use of the composition may and/or will provide one or more general health and/or general physiological benefits including, but not limited to, treatment of an upper gastrointestinal tract condition. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a package containing the composition). See e.g., the kits described herein.

As used herein, “treat,” “treating,” “treatment” or the like means that administration of the referenced composition prevents, alleviates, ameliorates, inhibits, or mitigates one or more symptoms of the condition or the condition itself, or any like benefit with respect to the gastrointestinal tract condition in a mammalian subject in need thereof, preferably in humans. As such, this includes, for example: preventing an upper gastrointestinal condition from occurring in a mammal, for example when the mammal is predisposed to acquiring the upper gastrointestinal condition, but has not yet been diagnosed with the disease; inhibiting the upper gastrointestinal condition; and/or alleviating, reversing, or curing the upper gastrointestinal condition. Insofar as the methods of the present invention are directed to preventing an upper gastrointestinal condition, it is understood that the term “prevent” does not require that the upper gastrointestinal condition be completely thwarted. Rather, as used herein, the term “preventing” or the like refers to the ability of the skilled artisan to identify a population that is susceptible to upper gastrointestinal conditions, such that administration of the referenced compositions may occur prior to the onset of the symptoms of the condition.

As used herein, one discontinuous schedule comprises, in the following order, a first dosing period, a first evaluation period, and a second dosing period. In an optional embodiment, the discontinuous schedule may comprise a second evaluation period and further, optionally, a third dosing period. In yet another embodiment, the discontinuous schedule may comprise even further alternating evaluation and dosing periods.

As used herein, “dosing period” refers to a period of time within the discontinuous schedule during which a unit dose is administered, preferably once daily. The dosing period may comprise at least about 2 days, more preferably from about 2 to about 28 days, even more preferably from about 5 to about 21 days, and most preferably from about 7 to about 14 days. The number of unit doses administered during each dosing period may be at least two, more preferably from 2 to about 28, more preferably from about 5 to about 21, and most preferably from about 7 to about 14. Optionally an identical number of unit doses is administered during each dosing period. Alternatively or additionally, it is optional that a unit dose is administered on consecutive days. However, it is neither required that an identical number of unit doses is administered during each dosing period (meaning, the dosing periods are independent of each other), nor that a unit dose be administered on consecutive days. The dosing periods begin with the administration of the first unit dose, and end upon administration of the last unit dose. Each dosing period, therefore, need not, but may be, identical in length.

As used herein, “evaluation period” refers to a period within a discontinuous schedule during which no unit doses are administered. An optional purpose of the evaluation period is to ensure that the gastric acid secretion inhibitor is effectively administered for over-the-counter use. Another purpose of the evaluation period is to give the user the opportunity to assess his or her symptoms. If symptoms do not recur, then the user may conclude that no additional dosing period is required. A given evaluation period comprises at least two days, more preferably from about 45 days to about 135 days, more preferably from about 75 days to about 135 days, and most preferably from about 90 days to about 125 days.

In one embodiment of this aspect, the discontinuous schedule comprising a first dosing period, a first evaluation period, a second dosing period, and a second evaluation period. In this embodiment, the first evaluation period is subsequent to the first dosing period and precedes the second dosing period, the second dosing period is subsequent to the first evaluation period and precedes the second evaluation period, and the second evaluation period is subsequent to the second dosing period and precedes any third dosing period or any other dosing or evaluation periods. Further dosing periods, alternating between evaluation periods are optional. Further evaluation periods, alternating between dosing periods are optional. The duration of all dosing and evaluation periods may be independent relative to the length of time of any other period.

As used herein, “subsequent to” refers to directly following the prior dosing period in time. As used herein, “preceding” refers to being directly prior to the time during which a new dosing period may begin. For example, administration of the final unit dose of a composition during a given dosing period marks the end of such dosing period and the beginning of the evaluation period.

Therefore, one embodiment of a discontinuous schedule provides for a first dosing period during which from about 10 to about 14 unit doses of a composition are administered, a first evaluation period from about 90 to about 125 days, and a second dosing period during which from about 10 to about 14 unit doses of a composition are administered.

Another embodiment herein provides for a first dosing period of about 10 to about 14 days, during which a total of about 10 to about 14 unit doses are administered individually once daily, followed by a first evaluation period from about 90 to about 125 days, followed by a second dosing period of about 10 to about 14 days, during which a total of about 10 to about 14 unit doses are administered individually once daily.

Another embodiment herein provides for a first dosing period of about 10 to about 14 days, during which a total of about 10 to about 14 unit doses are administered individually once daily, followed by a first evaluation period from about 90 to about 125 days, followed by a second dosing period of about 10 to about 14 days, during which a total of about 10 to about 14 unit doses are administered individually once daily, followed by a second evaluation period from about 90 to about 125 days, followed by a third dosing period of about 10 to about 14 days, during which a total of about 10 to about 14 unit doses are administered individually once daily.

### Examples

One example of a kit in accordance with the present invention includes an outer container in the form of a box. The outer container contains two separate inner containers, each in the form of smaller boxes that together fit inside the outer container (e.g., the kit comprises “boxes within a box”). Each separate inner container in turn contains two blister packs, with 7 unit doses of an aforementioned composition per blister pack (for example, the composition may comprise about 20 mg of an omeprazole magnesium salt). Therefore, each separate inner container contains 14 unit doses of the composition, and the kit as a whole contains a total of 28 unit doses of the composition. Each separate inner container also contains a folded piece of paper with written instructions for compliance with the described treatment method (alternatively in this example, each separate inner container has these written instructions printed on a surface of the inner container). The instructions read, in part, as follows:

### Course of Treatment:

Take every day for 14 days.

Do not take more than 1 tablet a day.

You may repeat a 14-day course of therapy every 4 months.

A second example of a kit includes an outer container in the form of a box. The outer container contains three separate inner containers, each in the form of smaller boxes that together fit inside the outer container. Each separate inner container in turn contains two blister packs, with seven unit doses per blister pack. Therefore, each separate inner container contains 14 unit doses of an aforementioned composition per blister pack (for example, the composition may comprise about 20 mg of an omeprazole magnesium salt) and the kit contains a total of 42 unit doses of the composition. Each separate inner container also contains a folded piece of paper with written instructions for compliance with the described treatment method (alternatively in this example, each separate inner container has these written instructions printed on a surface of the inner container).

1. A method of facilitating compliance with an over-the-counter heartburn treatment regimen comprising:
a. providing an outer container enclosing 2 inner containers wherein each inner container contains a blister pack and the blister pack contains 14 unit doses of a composition comprising a therapeutically effective amount of omeprazole or an omeprazole salt and wherein the outer container and each inner container further comprise a surface and wherein instructions for compliance with the heartburn treatment regimen are printed on the surface;
b. administering a treatment wherein one unit dose of the composition every day for 14 days; and
c. repeating the treatment after a period of about 75 days to about 135 days.

2. The method of claim 1 wherein the outer container is intended for over-the-counter sale.

3. (canceled)

4. The method of claim 1 wherein the composition is in a form selected from the group consisting of tablets and capsules.

5. The method of claim 1 wherein the composition is omeprazole.

6. The method of claim 1 wherein the composition is an omeprazole salt selected from the group consisting of omeprazole lithium salts, omeprazole sodium salts, omeprazole potassium salts, omeprazole magnesium salts, omeprazole calcium salts, and mixtures thereof.

7. The method of claim 6 wherein the omeprazole salt is omeprazole magnesium.

8. The method of claim 1 wherein the period is 4 months.

9. The method of claim 1 wherein each inner container is a box.

10. The method of claim 9 wherein the outer container is a box.

11. A method of facilitating compliance with an over-the-counter heartburn treatment regimen comprising:
a. providing an outer container enclosing 3 inner containers wherein each inner container contains a blister pack and the blister pack contains 14 unit doses of a composition comprising a therapeutically effective amount of omeprazole or an omeprazole salt and wherein the outer container and inner containers each further comprise a surface and wherein instructions for compliance with the heartburn treatment regimen are printed on the surface;
b. administering a treatment wherein one unit dose of the composition every day for 14 days; and
c. repeating the treatment after a period of about 75 days to about 135 days.

12. The method of claim 11 wherein the outer container is intended for over-the-counter sale.

13. The method of claim 11 wherein each inner container comprises one blister pack and the blister pack comprises the 14 unit doses.

14. The method of claim 11 wherein the composition is in a form selected from the group consisting of tablets and capsules.

15. The method of claim 11 wherein the composition is omeprazole.

16. The method of claim 11 wherein the composition is an omeprazole salt selected from the group consisting of omeprazole lithium salts, omeprazole sodium salts, omeprazole potassium salts, omeprazole magnesium salts, omeprazole calcium salts, and mixtures thereof.

17. The method of claim 16 wherein the omeprazole salt is omeprazole magnesium.

18. The method of claim 11 wherein the period is 4 months.

19. The method of claim 11 wherein each inner container is a box.

20. The method of claim 19 wherein the outer container is a box.

21. The method of claim 1 wherein each inner container comprises one blister pack and the blister pack comprises the 14 unit doses.
